(19) United States
(12) Patent Application Publication
(10) Pub. No.: US 2016/0041185 A1
(43) Pub. Date: Feb. 11, 2016

(54) Methods of Detecting Complement Fixing and Non-Complement Fixing Antibodies and Systems for Practicing the Same

(71) Applicant: THE BOARD OF TRUSTEES OF THE LELAND STANFORD JUNIOR UNIVERSITY, Palo Alto, CA (US)

(72) Inventors: Dolly B. Tyan, Palomar Park, CA (US); Ge Chen, Los Angeles, CA (US)

(21) Appl. No.: 14/775,146

(22) PCT Filed: Mar. 13, 2014
(86) PCT No.: PCT/US14/26425

§ 371 Date: Sep. 11, 2015

Related U.S. Application Data
(60) Provisional application No. 61/785,808, filed on Mar. 14, 2013.

(51) Int. Cl. G01N 33/68

(52) U.S. Cl. CPC G01N 33/6854

(57) ABSTRACT

Provided are methods for simultaneously determining the presence or absence of complement-fixing antibody (CFAb) and non-complement-fixing antibody (non-CFAb) in a biological sample in a single reaction. The methods include mixing a cellular sample from a donor, a biological sample from a recipient, isolated human complement component C1q (C1q), and a labeled C1q binding agent (CBA), under conditions sufficient for recipient CFAb and non-CFAb, if present, to bind to donor cell surface antigen (Ag) to form antibody-Ag (Ab-Ag-C1q-CBA) complexes. The methods further include contacting the mixture with labeled an-ti-hIgG antibodies and labeled anti-CD antibodies, and detecting (e.g., semi-quantitatively) the presence or absence of the detectable labels bound to the antibody-Ag complexes to determine the presence or absence of CFAb and non-CFAb in a biological sample from the recipient. Systems and kits for practicing the subject methods are also provided.

### CROSS-REFERENCE TO RELATED APPLICATIONS

This application claims priority to the filing date of U.S. Provisional Patent Application Ser. No. 61/785,808, filed Mar. 14, 2013, the disclosure of which is incorporated herein by reference in its entirety.

### BACKGROUND

The complement system is a complex group of proteins in blood that, in concert with antibodies and other factors, plays an important role as a mediator of immune, allergic, immunochemical and immunopathological reactions. Activation of the complement system can result in a wide range of reactions such as opsonization and lysis of various kinds of cells, bacteria and protozoa, inactivation of viruses, and the direct mediation of inflammatory processes. Through the hormone-like activity of several of its components, the complement system can recruit and enlist the participation of other humoral and cellular effector systems. These in turn can induce directed migration of leukocytes, trigger histamine release from mast cells, and stimulate the release of lysosomal constituents from phagocytes.

The complement system consists of at least twenty distinct plasma proteins capable of interacting with each other, with antibodies, and with cell membranes. Many of these proteins, when activated, combine with other proteins to form enzymes to cleave and activate still other proteins in the system. The sequential activation of these proteins, known as the complement cascade, follows two main pathways; the classical pathway and the alternative pathway. Both pathways converge at C3 and use a common terminal trunk which leads to cell lysis, bacterial opsonization and lysis, or viral inactivation.

The classical pathway can be activated by antigen-antibody complexes, aggregated immunoglobulins and non-immunological substances such as DNA and trypsin-like enzymes. The classical pathway of activation involves, successively, four components denominated C1, C4, C2 and C3. These components can be grouped into two functional units: C1 or recognition unit; and C4, C2, and C3 or activation unit. Five additional components denominated C5, C6, C7, C8, and C9 define the membrane attack complex (MAC) forming the terminal trunk common to both pathways that leads to cell lysis. The alternate pathway utilizes Factor B and bypasses the C1-C4-C2 steps, activating at C3.

The classical pathway begins with the C1-complex, which consists of one molecule of C1q and two molecules of both C1r and C1s. Activation of the C1-complex is triggered either by C1q's binding to antibodies from classes M and G, complexed with antigens, or by binding of C1q to the surface of a pathogen. Both C1r and C1s are serine proteases. Binding of C1q leads to conformational changes in the C1q molecule, which in turn leads to the activation of the two C1r molecules, followed by activation of the C1s molecules. In order to prevent spontaneous activation of this cascade, C1r and C1s are inhibited by C1-inhibitor, a serine protease inhibitor. Once activated, the C1-complex binds to and cleaves C2 and C4, producing C2a and C4b. C2a and C4b then bind to form a C4b2a complex, known as C3-convertase. Production of C3-convertase leads to cleavage of C3 into C3a and C3b; the latter joins with C2a and C4b (the C30 convertase) to make C5 convertase, which is the initial component of MAC.

Study and measurement of the activation of a complement pathway can provide an indication of many possible biological conditions and/or disorders. The complement pathway has been implicated in the pathogenesis or symptomatology of a broad spectrum of human diseases and pathologic conditions. Such diseases include immune complex diseases of several types, autoimmune diseases, in particular systemic lupus erythematosus, and infectious diseases, such as those found to be involved in infections with gram negative bacteria, viruses, parasites, fungi, and various dermatologic, renal, and hematologic diseases. Moreover, complement is a potent mediator and diagnostic indicator of inflammation and rejection in organ transplants, and complement-fixing human leukocyte antigen (HLA) antibodies are associated with antibody-mediated rejection and graft failure (Yabu et al. (2011) Transplantation 91(3):342-7).

### SUMMARY

Provided are methods for simultaneously determining the presence or absence of complement-fixing antibody (CFAb) and non-complement-fixing antibody (non-CFAb) in a biological sample in a single reaction. The methods include mixing a cellular sample from a donor, a biological sample from a recipient, isolated human complement component C1q (C1q), and a labeled C1q binding agent (CBA), under conditions sufficient for recipient CFAb and non-CFAb, if present, to bind to donor cell surface antigen (Ag) to form antibody-Ag (Ab-Ag-C1q-CBA) complexes. The methods further include contacting the mixture with labeled anti-hIgG antibodies and labeled anti-CD antibodies, and detecting (e.g., semi-quantitatively) the presence or absence of the detectable labels bound to the antibody-Ag complexes to determine the presence or absence of CFAb and non-CFAb in a biological sample from the recipient. Systems and kits for practicing the subject methods are also provided.

### BRIEF DESCRIPTION OF THE DRAWINGS

The invention may be best understood from the following detailed description when read in conjunction with the accompanying drawings. Included in the drawings are the following figures:

FIG. 1 schematically illustrates a method for determining the presence or absence of complement-fixing antibody (CFAb) and non-complement-fixing antibody (non-CFAb) in a biological sample according to one embodiment of the present disclosure.

FIG. 2 schematically illustrates a method for determining the presence or absence of complement-fixing antibody (CFAb) and non-complement-fixing antibody (non-CFAb) in a biological sample according to one embodiment of the present disclosure.

FIG. 3 shows results from an experiment performed according to an embodiment of the present disclosure. A predefined negative and two positive sera were tested by cFXM. FIG. 3, panel A shows a serum negative by cFXM-IgG and -C1q on both T and B cells; FIG. 3, panel B shows a serum positive by cFXM-IgG and -C1q on both T and B cells; FIG. 3, panel C shows a positive cFXM-IgG serum that is negative by cFXM-C1q.

FIG. 4 depicts results from an experiment showing a correlation between cFXM-IgG and IgG-FXM. Twenty five T and B cell crossmatches were analyzed using the standard IgG-FXM and the cFXM-IgG in parallel. MCS=median channel shift. The linear regression analysis shows highly significant correlation coefficients between cFXM-IgG and IgG-FXM on both T cells (R2=0.9805; P<0.0001) and B cells (R2=0.9282; P<0.0001).

FIG. 5 shows results from an experiment performed according to an embodiment of the present disclosure. A predefined negative (AB) serum and a serum from a sensitized patient making HLA antibodies of known specificity were tested by cFXM. The serum from the sensitized patient was known to contain both CFAb and Non-CFAb reactive to HLA on the target cells. Both donor specific CFAb and Non-CFAb were detected on T and B cells in the patient serum in the cFXM assay. To summarize the validation study of cFXM, a total of 25 samples were tested by Combo-Flow Crossmatch (cFXM), standard Flow Crossmatch with IgG detection (IgG-FXM), and long incubation complement dependent cytotoxicity (CDC). The donor specific antibodies were confirmed by Luminex HLA Single Antigen Beads (LMX-IgG).

FIG. 6 depicts results from an experiment performed according to an embodiment of the present disclosure. The cFXM-IgG shows high concordance with the paired IgG-FXM (Concordance: 84% for T-XM and 80% for B-XM). There were 4 T-XM (#16, #18, #22, #23) and 5 B-XM (#4, #16, #18, #22, #23) which were only positive by cFXM-IgG. All had donor specific antibody proven by LMX-IgG.

FIG. 7 shows results from an experiment performed according to an embodiment of the present disclosure. The concordance between cFXM-C1q with CDC (Concordance: 68% for T-XM and 84% for B-XM) is lower for T cells. This is a function of the insensitivity of the CDC assay. cFXM-C1q showed higher sensitivity than CDC on 12 paired samples (8 T-XM and 4 B-XM) and donor specific antibody was identified by LMX-IgG in all 12 samples.

FIG. 8 shows results of a validation study on 25 samples tested by cFXM, IgG-FXM, and complement dependent cytotoxicity (CDC). The donor specific antibodies were confirmed by Luminex HLA Single Antigen Beads (LMX-IgG). T=T cells, B=B cells. Median Channel Shift (MCS) cutoffs (lowest positive) for flow cytometric methods were: cFXM (IgG T=60, B=80; C1q T=40, B=45), IgG-FXM (T=165, B=200). CDC (Positive=21% over background cell death). DSA=donor specific antibody. MFI=mean fluorescence intensity (on the single antigen beads).

### DEFINITIONS

The term “complement-fixing antibody” (or “CFAb”) refers to an antibody that binds specifically to an antigen and initiates the complement cascade of the immune system that provides for clearance of the antigen bearing target (e.g., cell) or pathogen from the organism. In general, a complement fixing antibody is an IgM or an IgG antibody that is recognized and specifically bound by complement factor C1q, complement factor C3 via an alternate pathway, or the like.

The C1q complement factor is a subunit of the C1 enzyme complex that activates the serum complement system. It is composed of 9 disulfide-linked dimers of the chains A, B, and C, which share a common structure consisting of an N-terminal non-helical region, a triple helical (collagenous) region, and a C-terminal globular head (Smith et al. Biochem. J. 1994. 301:249-256). C1q is involved in host defense, inflammation, apoptosis, autoimmunity, cell differentiation, organogenesis, hibernation and insulin-resistant obesity. Five strictly conserved residues have been identified in the C1q family (Kishore et al. Trends in Immunology 2004. 25(10):551-561). Each C1q domain exhibits a ten-stranded -sandwich fold with a jelly-roll topology, consisting of two five-stranded β-sheets (A′, A, H, C, F) and (B′, B, G, D, E), each made of antiparallel strands. In general, the C1q complement factor is present in the serum of animals and has both binding specificity and binding affinity for complement fixing antibodies, which are also present in the serum of the animal. Binding of C1q complement factor to complement fixing antibodies activates the complement cascade of the immune system.

By “autologous” is meant derived from the same patient sample as another element. For example, in reference to autologous C1q is meant that the C1q occurs in the same subject sample as the complement fixing antibodies.

By “exogenous” is meant an element that is not naturally derived from a particular organism. For example, in reference to exogenous C1q, it is meant that the C1q is derived from an organism or system different from the subject sample having the complement fixing antibodies.

An “affinity reagent” of the subject invention has an analyte binding domain, moiety, or component that has a high binding affinity for a target analyte. By high binding affinity is meant a binding affinity of at least about 10⁻⁴ M, usually at least about 10⁻⁶ M or higher, e.g., 10⁻⁹ M or higher. The affinity reagent may be any of a variety of different types of molecules, so long as it exhibits the requisite binding affinity for the target protein when present as tagged affinity ligand.

As such, the affinity reagent may be a small molecule or large molecule ligand. By small molecule ligand is meant a ligand ranging in size from about 50 to about 10,000 daltons, usually from about 50 to about 5,000 daltons and more usually from about 100 to about 1000 daltons. By large molecule is meant a ligand in size from about 10,000 daltons or greater in molecular weight.

Of particular interest as large molecule affinity ligands are antibodies, as well as binding fragments and mimetics thereof. Where antibodies are the affinity ligand, they may be derived from polyclonal compositions, such that a heterogeneous population of antibodies differing by specificity are each tagged with the same tag (e.g., fluorophore), or monoclonal compositions, in which a homogeneous population of identical antibodies that have the same specificity for the target protein are each tagged with the same tag (e.g., fluorophore). As such, the affinity ligand may be a monoclonal, oligoclonal, and/or polyclonal antibody. In yet other embodiments, the affinity ligand is an antibody binding fragment or mimetic, where these fragments and mimetics have the requisite binding affinity for the target protein. For example, antibody fragments, such as Fv, (Fab')₂, and Fab may be prepared by cleavage of the intact protein, e.g. by protease or chemical cleavage. Also of interest are recombinantly produced antibody fragments, such as single chain antibodies or scFvs, where such recombinantly produced antibody fragments retain the binding characteristics of the above antibodies. Such recombinantly produced antibody fragments generally include at least the VH and VL domains of the subject antibodies, so as to retain the binding characteristics of the subject antibodies. These recombinantly produced antibody fragments or mimetics of the subject invention may be readily prepared using any convenient methodology, such as the methodology disclosed in U.S. Pat. Nos. 5,851,829 and 5,965,371; the disclosures of which are herein incorporated by reference.

The above described antibodies, fragments and mimetics thereof may be obtained from commercial sources and/or prepared using any convenient technology, where methods of producing polyclonal antibodies, oligoclonal antibodies, monoclonal antibodies, fragments and mimetics thereof, including recombinant derivatives thereof, are known to those of the skill in the art.

By “epitope” is meant a site on an antigen to which specific B cells and/or T cells respond. The term is also used interchangeably with “antigenic determinant” or “antigenic determinant site.” An epitope can comprise 1 or more amino acids, such as three or more amino acids, in a spatial conformation unique to the epitope. Generally, an epitope includes at least 5 such amino acids and, more usually, consists of at least 8-10 such amino acids. Methods of determining spatial conformation of amino acids are known in the art and include, for example, X-ray crystallography and 2-dimensional nuclear magnetic resonance. Furthermore, the identification of epitopes in a given protein is readily accomplished using techniques well known in the art. See, e.g., Geysen et al., Proc. Natl. Acad. Sci. USA (1984) 81:3998-4002 (general method of rapidly synthesizing peptides to determine the location of immunogenic epitopes in a given antigen); U.S. Pat. No. 4,708,871 (procedures for identifying and chemically synthesizing epitopes of antigens); and Geysen et al., Molecular Immunology (1986) 23:709-715 (technique for identifying peptides with high affinity for a given antibody). Antibodies that recognize the same epitope can be identified in a simple immunoassay showing the ability of one antibody to block the binding of another antibody to a target antigen.

By “binds specifically” or “specifically binds” is meant high avidity and/or high affinity binding of an antibody to a specific antigen or epitope. Antibody binding to its epitope on a specific antigen is with a greater avidity and/or affinity than binding of the same antibody to different epitopes, particularly different epitopes that may be present in molecules in association with, or in the same sample, as a specific antigen of interest. Complement fixing antibodies may, however, have the same or similar avidity and/or affinity for various epitopes on different antigens of interest. As such, “binds specifically” or “specifically binds” is not meant to preclude a given complement fixing antibody from binding to more than one antigen of interest. Antibodies that bind specifically to a polypeptide of interest may be capable of binding other polypeptides at a weak, yet detectable, level (e.g., 10% or less of the binding shown to the polypeptide of interest). Such weak binding, or background binding, is readily discernible from the specific antibody binding to the polypeptide of interest, e.g., by use of appropriate controls.

By “detectably labeled ligand”, or “detectably labeled secondary ligand” is meant a ligand having an attached detectable label, where the ligand is capable of binding specifically to another compound. Examples of ligands include, but are not limited to, and antibody or an antibody fragment that retains binding specificity. The detectable label may be attached by chemical conjugation, but where the label is a polypeptide, it could alternatively be attached by genetic engineering techniques. Methods for production of detectably labeled proteins are well known in the art. Detectable labels may be selected from a variety of such labels known in the art, but normally are radioisotopes, chromophores, fluorophores, fluorochromes, enzymes (e.g., horseradish peroxidase), linker molecules or other moieties or compounds which either emit a detectable signal (e.g., radioactivity, fluorescence, color) or emit a detectable signal after exposure of the label to its substrate. Various detectable label/substrate pairs (e.g., horseradish peroxidase/diaminobenzidine, biotin/streptavidin, luciferase/luciferin), methods for labeling antibodies, and methods for using labeled secondary antibodies to detect an antigen are well known in the art. See, e.g., Harlow and Lane, eds. (Using Antibodies: A Laboratory Manual (1999) Cold Spring Harbor Laboratory Press, Cold Spring Harbor, N.Y.).

By “directly labeled C1q” is meant an exogenous C1q molecule having an attached detectable label (e.g., a biotin-labeled C1q). The detectable label may be attached by chemical conjugation, but where the label is a polypeptide, it could alternatively be attached by genetic engineering techniques. Methods for production of detectably labeled proteins are well known in the art. Detectable labels may be selected from a variety of such labels known in the art, but normally are radioisotopes, chromophores, fluorophores, enzymes (e.g., horseradish peroxidase), linker molecules or other moieties or compounds which either emit a detectable signal (e.g., radioactivity, fluorescence, color) or emit a detectable signal after exposure of the label to its substrate. Various detectable label/substrate pairs (e.g., horseradish peroxidase/diaminobenzidine, biotin/streptavidin, luciferase/luciferin), methods for labeling antibodies, and methods for using labeled secondary antibodies to detect an antigen are known in the art. See, e.g., Harlow and Lane, eds. (Using Antibodies: A Laboratory Manual (1999) Cold Spring Harbor Laboratory Press, Cold Spring Harbor, N.Y.).

By “isolated” is meant a compound of interest that is in an environment different from that in which the compound naturally occurs. “Isolated” is meant to include compounds that are within samples that are substantially enriched for the compound of interest and/or in which the compound of interest is partially or substantially purified. The term “isolated” encompasses instances in which compound is unaccompanied by at least some of the material with which it is normally associated in its natural state. For example, the term “isolated” with respect to a polypeptide generally refers to an amino acid molecule devoid, in whole or part, of sequences normally associated with it in nature; or a sequence, as it exists in nature, but having heterologous sequences in association therewith.

As used herein, “purified” means that the recited material comprises at least about 75% by weight of the total protein, with at least about 80% being preferred, and at least about 90% being particularly preferred. As used herein, the term “substantially pure” refers to a compound that is removed from its natural environment and is at least 60% free, preferably 75% free, and most preferably 90% free from other components with which it is naturally associated.

A “biological sample” as used herein refers to a sample of tissue or fluid isolated from a subject, which in the context of the invention generally refers to samples suspected of containing anti-HLA complement fixing antibodies, which samples, after optional processing, can be analyzed in an in vitro assay. Typical samples of interest include, but are not necessarily limited to, blood, plasma, serum, blood cells, urine, saliva, and mucous. Samples also include samples of in vitro cell culture constituents including but not limited to conditioned media resulting from the growth of cells and tissues in culture medium, e.g., recombinant cells, and cell components.

By “human leukocyte antigen” or “HLA” is meant the major histocompatibility complex, which spans approximately 3.5 million base pairs on the short arm of chromosome 6. It is divisible into 3 separate regions which contain the class I, the class II and the class III genes. In humans, the class I HLA complex is about 2000 kb long and contains about 20 loci. Within the class I region exist genes encoding the well characterized class I MHC molecules designated HLA-A, HLA-B and HLA-C. In addition, there are non-classical class I genes encoded by the HLA-E, HLA-F, HLA-G, HLA-H, HLA-J, HLA-X and MIC loci. The class II region contains six gene families encoded by the HLA-DP, HLA-DQ and HLA-DRB1,3,4,5 loci. These genes encode the α and β chains of the classical class II MHC molecules designated HLA-DRB1,3,4,5, DP and DQ. In humans, non-classical genes encoded by the DM, DN and DO loci have also been identified within the class II region. The class III region contains a heterogeneous collection of more than 36 loci.

The terms “determining”, “measuring”, “evaluating”, “assessing” and “assaying” are used interchangeably and include quantitative and qualitative determinations.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### DETAILED DESCRIPTION

Provided are methods for simultaneously determining the presence or absence of complement-fixing antibody (CFAb) and non-complement-fixing antibody (non-CFAb) in a biological sample in a single reaction. The methods include mixing a cellular sample from a donor, a biological sample from a recipient, isolated human complement component C1q (C1q), and a labeled C1q binding agent (CBA), under conditions sufficient for recipient CFAb and non-CFAb, if present, to bind to donor cell surface antigen (Ag) to form antibody-Ag (Ab-Ag-C1q-CBA) complexes. The methods further include contacting the mixture with labeled anti-hIgG antibodies and labeled anti-CD antibodies, and detecting (e.g., semi-quantitatively) the presence or absence of the detectable labels bound to the antibody-Ag complexes to determine the presence or absence of CFAb and non-CFAb in a biological sample from the recipient. Systems and kits for practicing the subject methods are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an electrode” includes a plurality of such electrodes and reference to “the signal” includes reference to one or more signals, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflict with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods

As summarized above, aspects of the invention include methods for simultaneously determining the presence or absence of complement-fixing antibody (CFAb) and non-complement-fixing antibody (non-CFAb) in a biological sample in a single reaction. The methods include mixing a cellular sample from a donor, a biological sample from a recipient, isolated human complement component C1q (C1q), and a labeled C1q binding agent (CBA), under conditions sufficient for recipient CFAb and non-CFAb, if present, to bind to donor cell surface antigen (Ag) to form antibody-Ag (Ab-Ag-C1q-CBA) complexes. The methods further include contacting the mixture with labeled anti-hIgG antibodies and labeled anti-CD antibodies, and detecting (e.g., semi-quantitatively) the presence or absence of the detectable labels bound to the antibody-Ag complexes to determine the presence or absence of CFAb and non-CFAb in a biological sample from the recipient. Systems and kits for practicing the subject methods are also provided.

By “donor” is meant a source (e.g., a human source) of the cellular sample. The donor may be different from the recipient (e.g., where the CFAbs may be alloantibodies), or the donor and recipient may be the same (e.g., where the CFAbs may be autoantibodies). In certain aspects, the donor may be a candidate for donating cells (e.g., blood cells), tissues (e.g., cornea, skin, bone, heart valve, tendon, femoral and/or saphenous veins, lymph nodes, spleen, and the like), organs (e.g., a kidney, heart, liver, pancreas, lung, intestine, eye, and the like), and any combinations thereof, to a recipient in need thereof. Donors of interest include human donors, non-human primate donors, mammalian donors (e.g., pigs), non-mammalian donors, and any other donor types of interest.

As used herein, a “cellular sample” from a donor is a sample obtained from the donor that includes at least one cell. The at least one cell may be a nucleated cell (e.g., a lymphocyte or peripheral blood mononuclear cell (PBMC)), or a cell lacking a nucleus (e.g., an erythrocyte or platelet). In certain aspects, the cellular sample is a sample obtained from the donor that includes cells selected from lymphocytes (e.g., T cells, B cells, and/or natural killer (NK) cells), neutrophils, PBMCs, erythrocytes, platelets, monocytes, endothelial cells, and any combination thereof. According to certain embodiments, the cellular sample from the donor is from a tissue of the donor (e.g., lymph nodes, spleen, cornea, skin, bone, heart valve, tendon, femoral and/or saphenous veins, and the like), from an organ of the donor (e.g., a kidney, heart, pancreas, lung, liver, intestine, eye, and the like), or any combination of such tissues and/or organs. The cellular sample may be subjected to a purification procedure prior to use in the methods of the present disclosure. For example, the cellular sample may be a substantially pure sample of lymphocytes, peripheral blood mononuclear cells (PBMCs), erythrocytes, and/or platelets, which sample is free of components that may interfere with the mixing, contacting and/or detecting steps of the subject methods. In certain aspects, the subject methods include obtaining the cellular sample from the donor.

In certain aspects, the cellular sample from the donor includes from 0.1×10⁶ to 0.5×10⁶ cells. According to certain embodiments, the cellular sample from the donor includes 1×10⁶ or fewer cells, such as 0.5×10⁶ or fewer cells, 0.4×10⁶ or fewer cells, 0.3×10⁶ or fewer cells, 0.2×10⁶ or fewer cells, 0.1×10⁶ or fewer cells, 0.09×10⁶ or fewer cells, or fewer than 0.08×10⁶ cells. In certain aspects, the cellular sample from the donor includes 0.1×10⁶ or fewer cells.

By “recipient” is meant a source of the biological sample. The recipient may be different from the donor (e.g., where the CFAbs may be alloantibodies), or the recipient and donor may be the same (e.g., where the CFAbs may be autoantibodies). In certain aspects, the recipient (e.g., a human recipient) may be a candidate for receiving cells (e.g., blood cells), tissues (e.g., cornea, skin, bone, heart valve, tendon, femoral and/or saphenous veins, and the like), organs (e.g., a kidney, heart, pancreas, lung, liver, intestine, eye, and the like), and any combinations thereof, from the donor (e.g., to alleviate a medical condition) or may have already received cells, a tissue, or an organ from the donor. Recipients of interest include human recipients, non-human primate recipients, mammalian recipients, non-mammalian recipients, and any other recipient types of interest.

The “biological sample” from the recipient may be any biological sample from the recipient which includes or may include complement-fixing antibodies (CFAb) and/or non-complement fixing antibodies (non-CFAb). According to certain embodiments, the biological sample from the recipient is serum. In certain aspects, the biological sample from the recipient is 100 μL or less of serum, 90 μL or less of serum, 80 μL or less of serum, 70 μL or less of serum, 60 μL or less of serum, 50 μL or less of serum, 40 μL or less of serum, 30 μL or less of serum, 20 μL or less of serum, or 10 μL or less of serum. According to one embodiment, the biological sample from the recipient is 30 μL or less of serum. In certain aspects, the subject methods include obtaining the biological sample from the recipient.

In certain aspects, the isolated human complement component C1q (C1q) is unmodified, e.g., not conjugated to a detection moiety (e.g., not conjugated to biotin, a fluorophore, or the like). According to the present disclosure, unmodified C1(and the CFAbs to which the C1q binds) may be detected, e.g., using a detectably-labeled anti-C1q antibody (e.g., an anti-C1q antibody labeled with R-Phycoerythrin (PE) or any other detectable label (e.g., fluorophore) suitable for the assay being employed). In other aspects, the isolated human C1q is a modified human C1q that includes a moiety (such as biotin, a fluorophore, or any other moiety of interest) attached directly to the human C1q. For example, when the C1q is an isolated biotin labeled C1q (Bio-C1q), the C1q may be detected using a labeled agent that binds the biotin portion of the Bio-C1q (e.g., a fluorophore-labeled streptavidin, such as PE-labeled streptavidin or a streptavidin labeled with any other detectable label (e.g., fluorophore) suitable for the assay of interest.

Isolated biotin labeled C1q (Bio-C1q) is commercially available or may be obtained, e.g., by purifying C1q (e.g., human C1q) and biotinylating the purified protein. The purification of C1q is described, e.g., in Kishore et al. (1997) Biochem. J. 322:543. The purity of C1q may be assessed, e.g., by SDS-PAGE (15% w/v) under reducing conditions where it appears as three bands, corresponding to the A, B, and C chains of 34, 32, and 27 kDa, respectively. Biotinylation of C1q may be carried out, e.g., using a biotinylation kit (e.g., such as the EZ-Link Sulfo-NHS-LC Biotinylation Kit (Pierce, Rockford, Ill.)) as described, e.g., in Kishore et al. (2003) J. Immunology 171(2):812-820. R-Phycoerythrin-conjugated streptavidin (SA-PE) is commercially available from Life Technologies (Carlsbad, Calif.), Sigma-Aldrich (St. Louis, Mo.), and the like.

As noted above, the cellular sample from the donor, the biological sample from the recipient, the C1q, and the CBA are combined under conditions sufficient for recipient CFAb and non-CFAb, if present, to bind to donor cell surface antigen (Ag) to form antibody-Ag complexes (Ab-Ag) and complexes of Ab-Ag-C1q-CBA. In certain aspects, the cellular sample, the biological sample, the C1q, and the CBA are mixed concurrently. Conditions sufficient to permit the CFAb and non-CFAb, if present, to bind to donor cell surface antigen may be provided by selection of a suitable buffer (e.g., PBS, TBS, or the like), detergents (e.g., Tween), protein (e.g., BSA), pH, temperature, duration and/or the like. Conditions useful to permit specific binding of antibodies to their target antigens are described, e.g., in Coligan, et al., eds., Current Protocols in Immunology, John Wiley & Sons, Inc., N.Y. (1994-2013). In certain aspects, the cellular sample from the donor (e.g., 0.1×10⁶ cells), the biological sample from the recipient (e.g., 30 μL serum from the recipient), and 5 μL Bio-C1q/SA-PE mix are mixed for 30 minutes at room temperature in a suitable buffer to permit the CFAb and non-CFAb, if present, to bind to donor cell surface antigen. In certain aspects, the mixing, contacting and/or detecting steps are performed at room temperature.

In certain aspects, the mixture is washed prior to the contacting step. For example, all or substantially all of the unbound C1q, CBA, CFAb, and non-CFAb may be washed from the antibody-Ag complexes prior to contacting the mixture with labeled anti-hIgG and labeled anti-CD antibodies. Alternatively, or additionally, the mixture may be washed prior to the detecting step. The mixture may be washed one or more times (e.g., 1 or more, 2 or more, 3 or more, 4 or more, or 5 or more times) prior to the contacting step and/or detecting step. Washing finds use in reducing or eliminating any background signals which may otherwise be present when detecting the presence or absence of the detectable labels bound to the antibody-Ag complexes. Suitable wash buffers include, but are not limited to, a PBS- or TBS-based buffer which may include other components such as a detergent (e.g. Tween 20), bovine serum albumin, or any other useful components for washing the mixture prior to the contacting step and/or detecting step. Various wash buffers that find use in practicing the subject methods include those described, e.g., in Coligan, et al., eds., Current Protocols in Immunology, John Wiley & Sons, Inc., NY (1994-2013).

When the cellular sample includes or is suspected of including B cells, the methods of the present disclosure may include adding an effective amount of one or more proteolytic enzymes under conditions sufficient to remove any C1q receptors from the B cells. In certain aspects, the one or more proteolytic enzymes is a group of proteolytic enzymes that are produced in the culture supernatant of Streptomyces griseus K-1, such as pronase (available from, e.g., EMD Millipore (Bellerica, Mass.)). The pronase may be added to the cellular sample prior to combining the cellular sample with the biological sample from the recipient, the C1q, and the CBA. Alternatively, or additionally, the pronase may be added after combining the cellular sample with the biological sample from the recipient, the C1q, and the CBA. In certain aspects, the effective amount is an amount of pronase added such that the final concentration of the pronase in the cellular sample or mixture is from 0.01 mg/ml to 5 mg/ml, e.g., 0.25 mg/ml.

The contacting step may include combining the labeled anti-hIgG antibodies and labeled anti-CD antibodies with the mixture under conditions sufficient to permit the labeled antibodies to bind hIgG present in the antibody-Ag complexes and CD present on the cells, respectively. Such conditions may include those that find use in labeled secondary antibody incubation procedures, and may employ a PBS- or TBS-based buffer optionally including other components such as a detergent (e.g. Tween 20), bovine serum albumin, and/or any other components useful to permit the labeled antibodies to bind hIgG and CD present in the antibody-Ag complexes and on the cells. In certain aspects, the anti-CD antibodies include antibodies that specifically bind a CD (“cluster of differentiation”) antigen present on the surface of T cells (e.g., CD3, CD4, and/or the like), a CD antigen present on the surface of B cells (e.g., CD19, CD20, CD21, and/or the like), or a mixture that includes antibodies that specifically bind to a CD present on T cells and antibodies that specifically bind to a CD present on B cells, where the T cell- and B cell-specific antibodies are optionally distinguishable from each other by virtue of having differentially detectable labels (e.g., Per-CP-CD3 and APC-CD19 to detect T cell and B cells, respectively, in the same assay). In certain aspects, the contacting step occurs for less than one hour (e.g., about 30 minutes).

The methods of the present disclosure include detecting the presence or absence of the detectable labels bound to the antibody-Ag complexes to determine the presence or absence of CFAb and non-CFAb in a biological sample from the recipient. The detection strategy employed may vary according to the types of detectable labels present in the labeled anti-hIgG antibodies and labeled anti-CD antibodies. Detectable labels that find use in practicing the subject methods include, but are not limited to, a fluorophore, a chromophore, an enzyme, a linker molecule, a biotin molecule, an electron donor, an electron acceptor, a dye, a metal, or a radionuclide. In certain aspects, the labeled anti-hIgG and labeled anti-CD antibodies comprise different labels (e.g., differentially detectable labels).

According to certain embodiments, the labeled anti-hIgG and/or labeled anti-CD antibodies are fluorescently-labeled and comprise a fluorophore selected from indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, Alexa Fluor 700, JOE, Lissamine, Rhodamine Green, BODIPY, fluorescein isothiocyanate (FITC), carboxy-fluorescein (FAM), Allophycocyanin (APC), phycoerythrin (PE), rhodamine, dichlororhodamine (dRhodamine), carboxy tetramethylrhodamine (TAMRA), carboxy-X-rhodamine (ROX), LIZ, VIC, NED, PET, SYBR, PicoGreen, and RiboGreen.

When the labeled anti-hIgG and/or labeled anti-CD antibodies are fluorescently-labeled, the detecting may include detecting one or more fluorescence emissions. The fluorescence emission(s) may be detected in any useful format. In certain aspects, the detecting includes flowing the mixture (e.g., a mixture that includes the antibody-Ag complexes with bound labeled anti-hIgG antibodies and bound labeled anti-CD antibodies) through a flow cytometer.

When the detecting includes flowing the mixture through a flow cytometer, the flow cytometer is configured to detect and uniquely identify the antibody-Ag complexes by exposing the complexes to excitation light and measuring the fluorescence of each complex in one or more detection channels, as desired. The excitation light may be from one or more light sources and may be either narrow or broadband. Examples of excitation light sources include lasers, light emitting diodes, and arc lamps. Fluorescence emitted in detection channels used to identify the complexes may be measured following excitation with a single light source, or may be measured separately following excitation with distinct light sources. In certain aspects, the flow cytometer through which the mixture is flowed includes fluorescence excitation and detection capabilities such that the fluorescent label of the anti-IgG antibodies, the phycoerythrin (PE) label of the SA-PE, and the fluorescent label of the anti-CD antibodies of the antibody-Ag complexes are each detectable and distinguishable upon interrogation of the complexes by the flow cytometer.

Flow cytometers further include data acquisition, analysis and recording means, such as a computer, wherein multiple data channels record data from each detector for the light scatter and fluorescence emitted by each complex as it passes through the sensing region. The purpose of the analysis system is to classify and count complexes where each complex presents itself as a set of digitized parameter values. The flow cytometer may be set to trigger on a selected parameter in order to distinguish the complexes of interest from background and noise. “Trigger” refers to a preset threshold for detection of a parameter. It is typically used as a means for detecting passage of a complex through the laser beam. Detection of an event which exceeds the threshold for the selected parameter triggers acquisition of light scatter and fluorescence data for the complex. Data is not acquired for complexes or other components in the medium being assayed which cause a response below the threshold. The trigger parameter may be the detection of forward scattered light caused by passage of a complex through the light beam. The flow cytometer then detects and collects the light scatter and fluorescence data for the complex.

Flow cytometric analysis of the complexes, as described above, yields qualitative and quantitative information about the complexes. Where desired, the above analysis yields counts of the complexes of interest in the mixture. As such, the flow cytometric analysis provides data regarding the numbers of one or more different types of complexes in the mixture.

The mixing, contacting and detecting steps may be performed collectively in any convenient amount of time. According to certain embodiments, the methods of the present disclosure are performed in 5 hours or less, 4 hours or less, 3 hours or less (e.g., about 2.5 hours), or 2 hours or less.

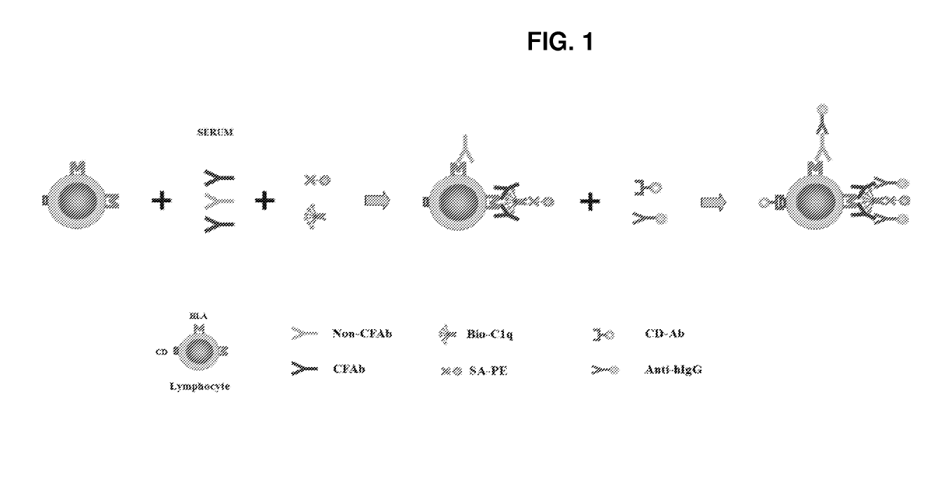

A method (designated “combo flow crossmatch” or “cFXM”) according to one embodiment of the present disclosure is schematically illustrated in FIG. 1. As shown, a lymphocyte of a cellular sample from a donor having a CD antigen and two HLA antigens is mixed with: a biological sample (in this example, serum) from a recipient which includes complement-fixing antibodies (CFAbs, the darker colored antibodies as shown) and non-complement-fixing antibodies (non-CFAbs, the lighter antibody as shown); isolated human biotin labeled C1q (Bio-C1q); and R-Phycoerythrin-conjugated streptavidin (SA-PE). The mixing is carried out under conditions sufficient for the CFAbs and the non-CFAbs to bind the donor cell surface antigens (in this example, the HLA antigens on the surface of the donor lymphocyte), thereby forming an antibody-donor cell surface antigen (antibody-Ag) complex. In this example, the complex also includes Bio-C1q bound to the CFAbs, and SA-PE bound to the Bio-C1q by virtue of the biotin-streptavidin interaction. Bio-C1q does not bind to the non-CFAbs. The antibody-Ag complex is then contacted with labeled anti-hIgG and labeled anti-CD antibodies. The labeled anti-hIgG antibodies serve as secondary antibodies which bind both the CFAbs and non-CFAbs. The labeled anti-CD antibody binds the CD antigen on the surface of the lymphocyte in the complex. The antibody-Ag complex with bound labeled anti-hIgG and labeled anti-CD antibodies is shown on the far right of the upper panel of FIG. 1. In this “final” complex, both the CFAbs and non-CFAbs may be detected via the detectable label of the labeled anti-hIgG antibodies, the CFAbs (and not the non-CFAbs) may be detected via the PE moiety of the SA-PE, and the identity of the lymphocyte (e.g., T cell or B cell) may be determined by detecting a T cell- or B cell-specific label of the labeled anti-CD antibody. Each type of label present in each complex may be detected, e.g., by flowing the mixture that includes the complexes through a flow cytometer. Complexes may be counted based on the presence of CFAbs, non-CFAbs, CFAbs and non-CFAbs, the type of lymphocyte (e.g., T cell or B cell), or any combination thereof. Moreover, the detection of any of the labels may be quantitative, e.g., such that the intensity of the CFAb-specific signal (from the PE portion of the SA-PE) is determined to provide information with respect to the amount/concentration of CFAbs in the biological sample from the recipient.

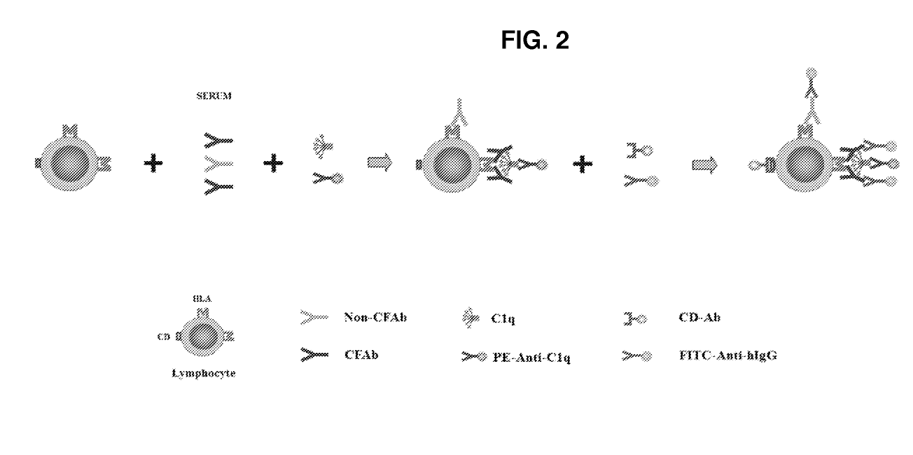

A cFXM method according to an embodiment of the present disclosure is schematically illustrated in FIG. 2. As shown, a lymphocyte of a cellular sample from a donor having a CD antigen and two HLA antigens is mixed with: a biological sample (in this example, serum) from a recipient which includes complement-fixing antibodies (CFAbs, the darker colored antibodies as shown) and non-complement-fixing antibodies (non-CFAbs, the lighter antibody as shown); isolated human C1q; and a PE-labeled anti-C1q antibody. The mixing is carried out under conditions sufficient for the CFAbs and the non-CFAbs to bind the donor cell surface antigens (in this example, the HLA antigens on the surface of the donor lymphocyte), thereby forming an antibody-donor cell surface antigen (antibody-Ag) complex. In this example, the complex also includes human C1q bound to the CFAbs, and PE-labeled anti-C1q antibody bound to the C1q by virtue of the specific binding of the PE-labeled anti-C1q antibody to the human C1q. The human C1q does not bind to the non-CFAbs. The antibody-Ag complex is then contacted with labeled anti-hIgG and labeled anti-CD antibodies. The labeled anti-hIgG antibodies serve as secondary antibodies which bind both the CFAbs and non-CFAbs. The labeled anti-CD antibody binds the CD antigen on the surface of the lymphocyte in the complex. The antibody-Ag complex with bound labeled anti-hIgG and labeled anti-CD antibodies is shown on the far right of the upper panel of FIG. 2. In this “final” complex, both the CFAbs and non-CFAbs may be detected via the detectable label of the labeled anti-hIgG antibodies, the CFAbs (and not the non-CFAbs) may be detected via detection of the PE moiety of the PE-labeled anti-C1q antibody, and the identity of the lymphocyte (e.g., T cell or B cell) may be determined by detecting a T cell- or B cell-specific label of the labeled anti-CD antibody. Each type of label present in each complex may be detected, e.g., by flowing the mixture that includes the complexes through a flow cytometer. Complexes may be counted based on the presence of CFAbs, non-CFAbs, CFAbs and non-CFAbs, the type of lymphocyte (e.g., T cell or B cell), or any combination thereof. Moreover, the detection of any of the labels may be quantitative, e.g., such that the intensity of the CFAb-specific signal (from the PE portion of the PE-labeled anti-C1q antibody) is determined to provide information with respect to the amount/concentration of CFAbs in the biological sample from the recipient.

### Systems

Also provided are systems for performing the methods of the present disclosure. Systems of the present disclosure include a sample fluid subsystem that includes a processor and a computer-readable medium operably coupled to the processor with stored programming thereon. When executed by the processor, the stored programming programs the processor to cause the sample fluidic subsystem to form a mixture by combining a cellular sample from a donor, a biological sample from a recipient, isolated human C1q (C1q), and a labeled C1q binding agent (CBA), under conditions sufficient for recipient CFAb and non-CFAb, if present, to bind to Ag to form antibody-Ag complexes, and contact the mixture with labeled anti-hIgG antibodies and labeled anti-CD antibodies. The systems of the present disclosure further include a flow cytometer configured to assay the sample for the presence or absence of the detectable labels bound to the antibody-Ag complexes to determine the presence or absence of CFAb and non-CFAb in a biological sample from the recipient. In certain aspects, the flow cytometer is fluidically coupled to the sample fluidic subsystem.

The processor may be any suitable processor for executing the stored programming. In certain aspects, the processor is programmed to cause the sample fluidic subsystem to mix the cellular sample, the biological sample, the C1q, and the CBA concurrently. According to certain embodiments, the processor is programmed to cause the sample fluidic subsystem to wash the mixture before the subsystem contacts the mixture with the labeled anti-CD and labeled anti-hIgG antibodies. Alternatively, or additionally, the processor may be programmed to cause the sample fluidic subsystem to wash the mixture after the subsystem contacts the mixture with the labeled anti-CD and labeled anti-hIgG antibodies, but before the flow cytometer assays the sample for the presence or absence of the detectable labels bound to the complexes. In certain aspects, the processor is programmed to cause the sample fluidic subsystem to add an effective amount of pronase under conditions sufficient to remove C1q receptor from any B cells present in the cellular sample.

The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. A computer readable storage medium may be any tangible medium that can contain, or store a program for use by or in connection with the sample fluidic subsystem of the systems of the present disclosure.

The cellular sample from the donor, the biological sample from the recipient, the C1q, the CBA, the labeled anti-hIgG antibodies, the labeled anti-CD antibodies, and the flow cytometer may be as described hereinabove with respect to the methods of the present disclosure.

The systems of the present disclosure may be configured to detect the presence or absence of CFAb and non-CFAb in a convenient amount of time. According to certain embodiments, the subject systems are configured to detect the presence or absence of CFAb and non-CFAb in 5 hours or less, 4 hours or less, 3 hours or less, or 2 hours or less.

### Kits

Kits which include one or more reagents useful for performing the methods of the present disclosure are also provided. According to one embodiment, provided is a kit that includes a reagent selected from isolated human C1q, isolated CBA, labeled anti-CD antibodies, labeled anti-hIgG antibodies, or any combination of such reagents (e.g., each of these reagents), as well as instructions for using the C1q, CBA, labeled anti-CD antibodies and labeled anti-hIgG antibodies to assay (e.g., by flow cytometry) a cellular sample from a donor and a biological sample from a recipient to determine the presence or absence of CFAb and non-CFAb in the biological sample. The C1q, CBA, labeled anti-hIgG antibodies, and labeled anti-CD antibodies may be as described hereinabove with respect to the methods of the present disclosure.

Reagents included in the subject kits may be provided in separate tubes, or two or more reagents may be provided in a single tube. According to one embodiment, the C1q and CBA are provided in a single tube and/or the labeled anti-hIgG antibodies and labeled anti-CD antibodies are provided in a single tube.

According to one embodiment, instructions included in the subject kits are provided on a computer-readable medium which, when executed by a processor, programs the processor to assay a cellular sample from a donor and a biological sample from a recipient to determine the presence or absence of CFAb and non-CFAb in the biological sample.

### Utility

The subject methods, systems and kits find use in any application in which it is desirable to detect complement-fixing antibodies and non-complement fixing antibodies in a biological sample of a recipient. Recipients of interest include, but are not limited to, human recipients in need of, or having already received, an organ (e.g., kidney, liver, heart, etc.) or tissue transplant from an organ or tissue donor. Applications of interest include pre-transplantation risk assessment and/or post-transplantation monitoring based on detecting and/or quantifying the levels of CFAbs and/or non-CFAbs in the biological sample of the recipient. The methods, systems, and kits of the present disclosure constitute a marked improvement over previous methods, in that a universal platform for simultaneously detecting CFAb and non-CFAb in a single reaction is provided. Moreover, the methods, systems, and kits of the present disclosure enable a clinician or researcher to distinguish clinically relevant antibodies from non-clinically relevant antibodies, and dramatically reduces the time required to detect/quantitate CFAb and non-CFAb, where the duration of the assay may be critical to patient and/or organ survival in the context of organ or tissue transplantation.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Efforts have been made to ensure accuracy with respect to numbers used (e.g. amounts, temperature, etc.) but some experimental errors and deviations should be accounted for.

### Example 1

### Testing of Pre-Defined Negative and Positive Sera by cFXM

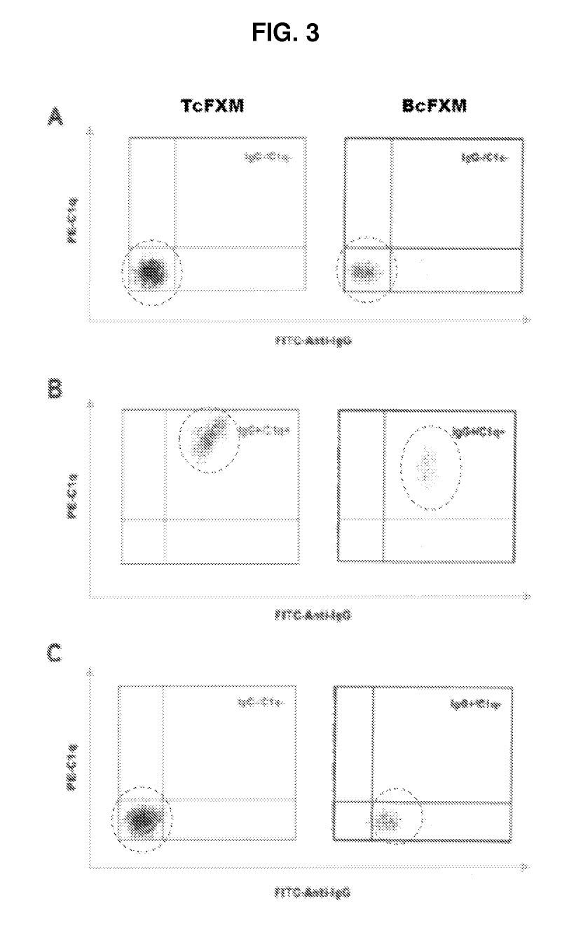

A predefined negative and two positive sera were tested by cFXM. FIG. 3, Panel A shows a serum negative by cFXM-IgG and -C1a on both T and B cells; FIG. 3, Panel B shows a serum positive by cFXM-IgG and -C1q on both T and B cells; FIG. 3, Panel C shows a positive cFXM-IgG serum that is negative by cFXM-C1q.

### Example 2

### Correlation Between cFXM-IgG and IgG-FXM

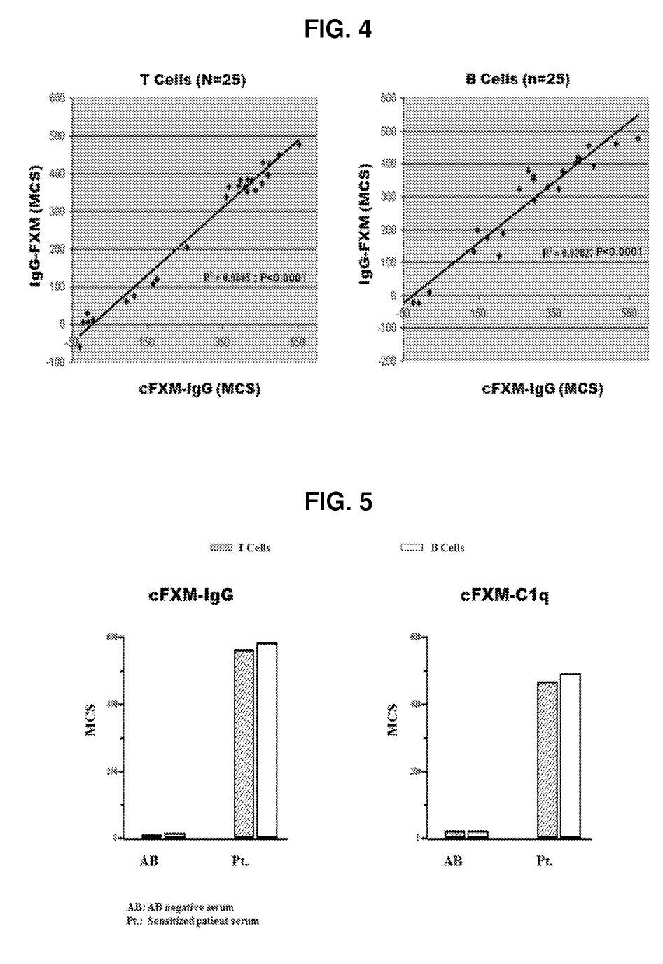

Twenty five T and B cell crossmatches were analyzed using the standard IgG-FXM and the cFXM-IgG in parallel. MCS=median channel shift. The linear regression analysis shows highly significant correlation coefficients between cFXM-IgG and IgG-FXM on both T cells (R2=0.9805; P<0.0001) and B cells (R2=0.9282; P<0.0001). Results are shown in FIG. 4.

### Example 3

### Testing of Predefined Negative (AB) Serum and a Serum from a Sensitized Patient Making HLA Antibodies or Known Specificity by cFXM

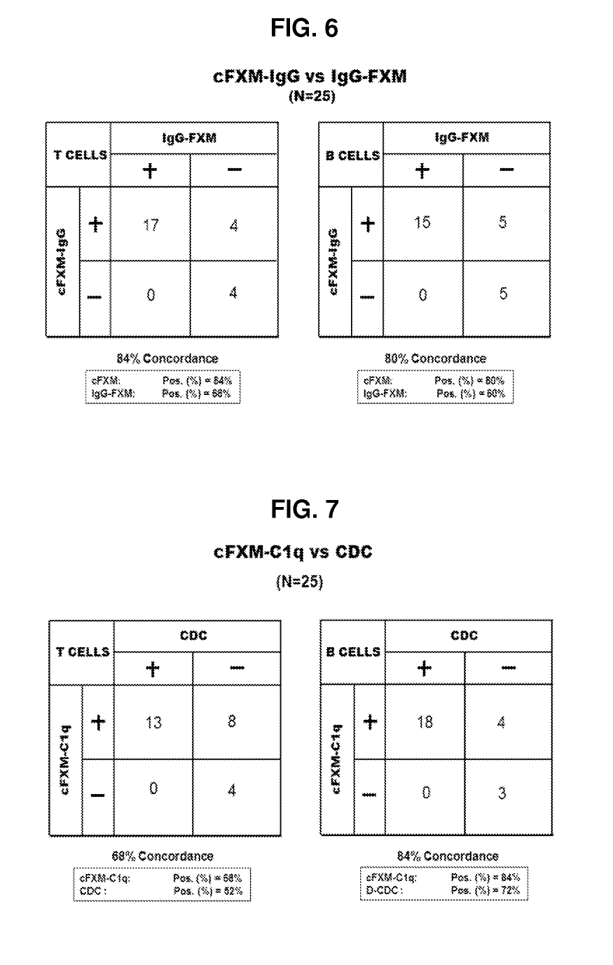

A predefined negative (AB) serum and a serum from a sensitized patient making HLA antibodies of known specificity were tested by cFXM. The serum from the sensitized patient was known to contain both CFAb and Non-CFAb reactive to HLA on the target cells. Both donor specific CFAb and Non-CFAb were detected on T and B cells in the patient serum in the cFXM assay. Results are shown in FIG. 5.

To summarize the above validation studies of cFXM, a total of 25 samples were tested by Combo-Flow Crossmatch (cFXM), standard Flow Crossmatch with IgG detection (IgG-FXM), and long incubation complement dependent cytotoxicity (CDC). The donor specific antibodies were confirmed by Luminex HLA Single Antigen Beads (LMX-IgG).

### Example 4

### cFXM-IgG Shows High Concordance with the Paired IgG-FXM

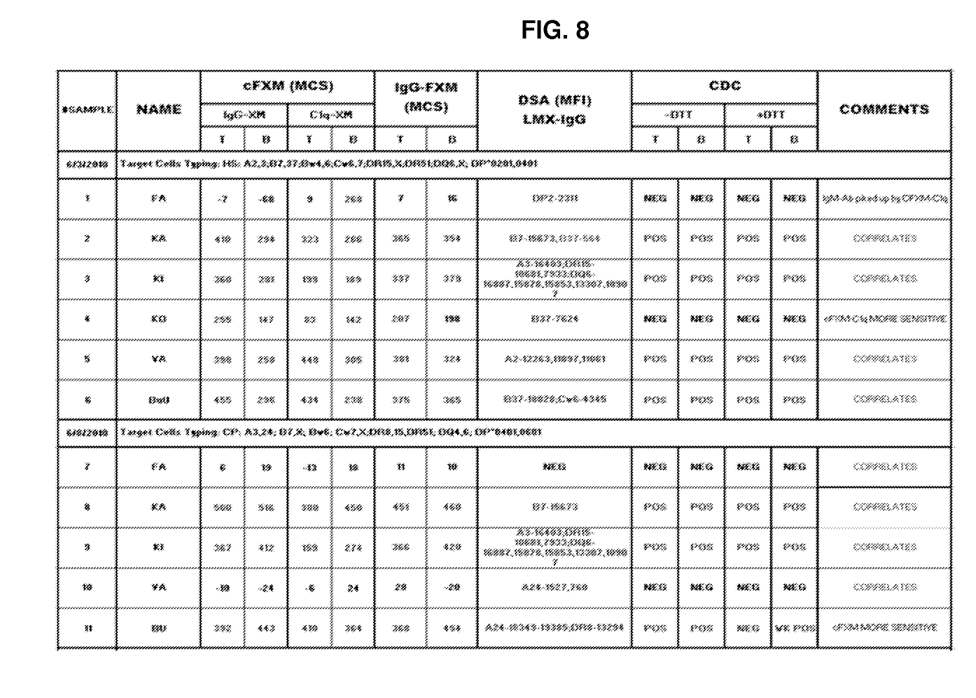

The cFXM-IgG shows high concordance with the paired IgG-FXM (Concordance: 84% for T-XM and 80% for B-XM). There were 4 T-XM (#16, #18, #22, #23) and 5 B-XM (#4, #16, #18, #22, #23) which were only positive by cFXM-IgG. All had donor specific antibody proven by LMX-IgG. Results are shown in FIG. 6.

### Example 5

### Concordance Between cFXM-C1q with CDC

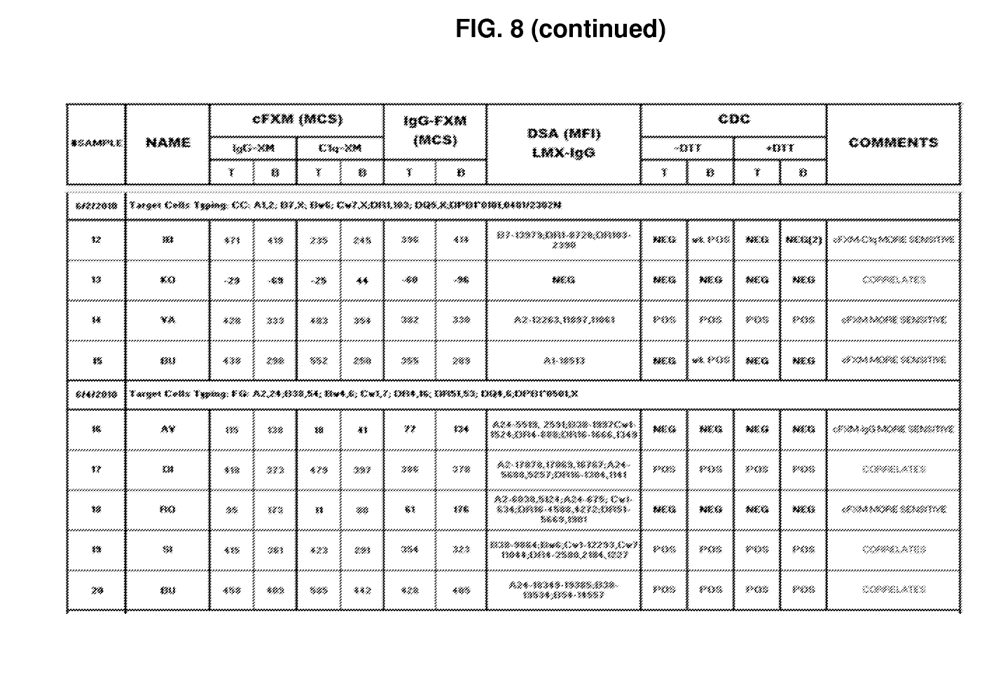

The concordance between cFXM-C1q with CDC (Concordance: 68% for T-XM and 84% for B-XM) is lower for T cells. This is a function of the insensitivity of the CDC assay. cFXM-C1q showed higher sensitivity than CDC on 12 paired samples (8 T-XM and 4 B-XM) and donor specific antibody was identified by LMX-IgG in all 12 samples. Results are shown in FIG. 7.

### Example 6

### Results of a Validation Study on Samples Tested by cFXM. IgG-FXM, and Complement Dependent Cytotoxcicity (CDC)

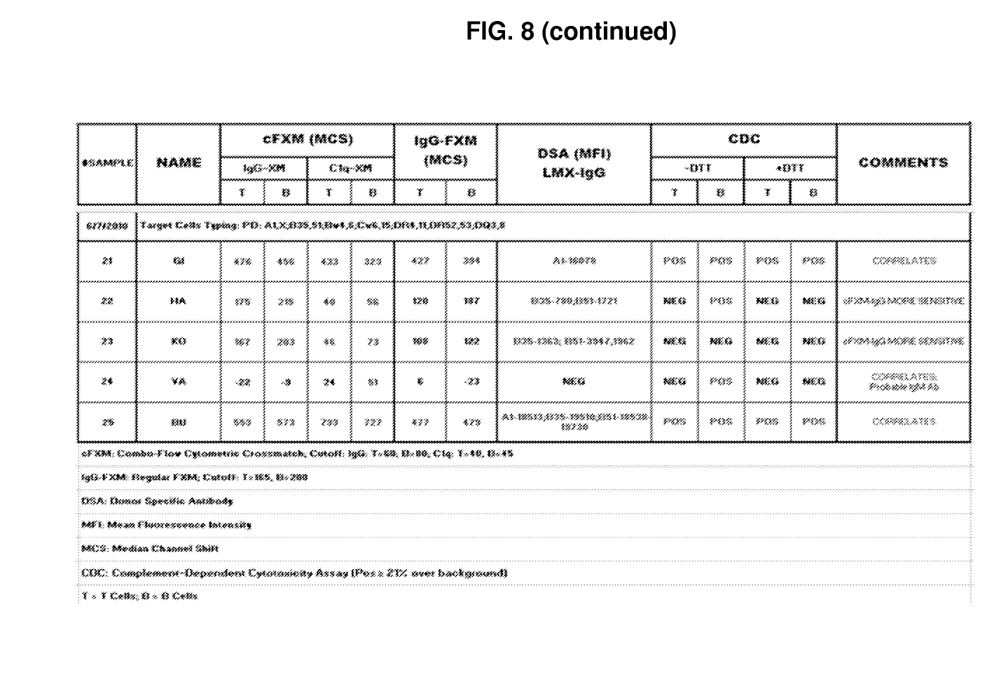

Results of validation study on 25 samples tested by cFXM, IgG-FXM, and complement dependent cytotoxicity (CDC). The donor specific antibodies were confirmed by Luminex HLA Single Antigen Beads (LMX-IgG). T=T cells, B═B cells. Median Channel Shift (MCS) cutoffs (lowest positive) for flow cytometric methods were: cFXM (IgG T=60, B=80; C1q T=40, B=45), IgG-FXM (T=165, B=200). CDC (Positive=21% over background cell death). DSA=donor specific antibody. MFI=mean fluorescence intensity (on the single antigen beads). Results are shown in FIG. 8.

### Example 7

### cFXM Using a Labeled Anti-C1q Antibody

In this example, 50 μl of recipient serum is incubated at 56° C. for 30 minutes to inactivate autologous complement. Purified human C1q is added to a final concentration of 150 μg/ml. 1×10⁶ donor peripheral blood mononuclear cells (PBMCs) are treated with 0.25 mg/ml pronase for 20 minutes to eliminate C1q receptors on the surfaces of B cells.

To 0.1×10⁶ donor pronase-treated donor PBMC cells, 30 μl of recipient serum and 5 μl PE-labeled anti-human C1q are added. After a 30 minute incubation at room temperature (23° C.), the cells are washed three times with 3% HBSA and resuspended in 100 μl mix of APC anti-IgG, FITC-CD3 and PerCP-CD19, followed by incubation at RT for an additional 20 minutes.

The cells are washed twice with 3% HBSA and resuspended in 200 μl of 0.2% paraformaldehyde in PBS. The sample is then loaded onto a flow cytometer (e.g., Canto-II) and 10,000 events are collected. The Median Channel Shift of test sera and controls is calculated.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method for simultaneously determining the presence or absence of complement-fixing antibody (CFAb) and non-CFAb in a biological sample in a single reaction, comprising:
forming a mixture by combining a cellular sample from a donor, a biological sample from a recipient, isolated complement component C1q (C1q), and a labeled C1q binding agent (CBA), under conditions sufficient for recipient CFAb and non-CFAb, if present, to bind to donor cell surface antigen (Ag) to form antibody-Ag complexes (Ab-Ag) and complexes of Ab-Ag-C1q-CBA;
contacting the mixture with labeled anti-hIgG antibodies and labeled anti-CD antibodies; and
detecting the presence or absence of the detectable labels bound to the antibody-Ag complexes to determine the presence or absence of CFAb and non-CFAb in a biological sample from the recipient.

2. The method according to claim 1, wherein the detecting is quantitative.

3. The method according to claim 1, wherein the cellular sample, the biological sample, the C1q, and the CBA are mixed concurrently.

4. The method according to claim 1, comprising washing the mixture before the contacting.

5. The method according to claim 1, comprising washing the mixture after the contacting and before the detecting.

6. The method according to claim 1, wherein the cellular sample comprises T cells.

7. The method according to claim 1, wherein the cellular sample comprises B cells.

8. The method according to claim 7, comprising adding an effective amount of pronase under conditions sufficient to remove C1q receptor from the B cells.

9. The method according to claim 8, wherein the final pronase concentration is from 0.01 mg/ml to 5 mg/ml.

10. The method according to claim 1, wherein the method is performed at room temperature.

11. The method according to claim 1, wherein the detecting comprises detecting a fluorescence emission.

12. The method according to claim 1, wherein the detecting comprises flowing the mixture through a flow cytometer.

13. The method according to claim 1, wherein the detectable label comprises a fluorochrome, a chromophore, an enzyme, a linker molecule, a biotin molecule, an electron donor, an electron acceptor, a dye, a metal, or a radionuclide.

14. The method according to claim 1, wherein the labeled anti-hIgG and labeled anti-CD antibodies comprise different labels.

15. The method according to claim 1, wherein the labeled anti-hIgG antibodies comprise a fluorophore selected from the group consisting of: indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, Alexa Fluor 700, JOE, Lissamine, Rhodamine Green, BODIPY, fluorescein isothiocyanate (FITC), carboxy-fluorescein (FAM), Allophycocyanin (APC), phycoerythrin (PE), rhodamine, dichlororhodamine (dRhodamine), carboxy tetramethylrhodamine (TAMRA), carboxy-Xrhodamine (ROX), LIZ, VIC, NED, PET, SYBR, PicoGreen, and RiboGreen.

16. The method according to claim 1, wherein the labeled anti-CD antibodies comprise a fluorophore selected from the group consisting of: indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, Alexa Fluor 700, JOE, Lissamine, Rhodamine Green, BODIPY, fluorescein isothiocyanate (FITC), carboxy-fluorescein (FAM), Allophycocyanin (APC), phycoerythrin (PE), rhodamine, dichlororhodamine (dRhodamine), carboxy tetramethylrhodamine (TAMRA), carboxy-Xrhodamine (ROX), LIZ, VIC, NED, PET, SYBR, PicoGreen, and RiboGreen.

17. The method according to claim 1, wherein the biological sample comprises serum.

18. The method according to claim 17, wherein the biological sample comprises 10 μl to 100 μL of serum.

19. The method according to claim 17, wherein the biological sample comprises 30 μL or less of serum.

20. The method according to claim 1, wherein the biological sample comprises plasma.

21. The method according to claim 1, comprising obtaining the cellular sample from the donor.

22. The method according to claim 1, comprising obtaining the biological sample from the recipient.

23. The method according to claim 1, wherein the cellular sample from the donor comprises from 0.1×10⁶ to 0.5×10⁶ cells.

24. The method according to claim 1, wherein the cellular sample from the donor comprises fewer than 0.1×10⁶ cells.

25. The method according to claim 1, wherein the method is performed in 4 hours or less.

26. The method according to claim 1, wherein the anti-CD antibodies are selected from the group consisting of anti-CD3, anti-CD19, and anti-CD20.

27-38. (canceled)
